(19) United States
(12) Patent Application Publication
(10) Pub. No.: US 2017/0151147 A1
(43) Pub. Date: Jun. 1, 2017

(54) HIGH-PERFORMANCE HAIR TREATMENT AGENT WITH ANTI-WASH-OUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/359,006

(22) Filed: Nov. 22, 2016

(30) Foreign Application Priority Data
Dec. 1, 2015 (DE) 10 2015 223 840.7

(51) Int. Cl. A61K 8/49; A61K 8/41; A61K 8/73; A61Q 17/00; A61Q 5/12; A61K 8/36; A61Q 5/00; A61K 8/34; A61K 8/362

(52) U.S. Cl. CPC A61K 8/4953; A61K 8/342; A61K 8/34; A61K 8/416; A61K 8/737; A61K 8/362; A61Q 5/12; A61K 8/36; A61K 8/731; A61Q 5/004; A61Q 17/005; A61K 2800/596; A61K 2800/5426

(57) ABSTRACT

Hair treatment agents are provided that include at least one fatty alcohol, at least one cationic surfactant, hexetidine and phenoxyethanol, to reduce or prevent the washing-out of color from colored hair.

### CROSS-REFERENCE TO RELATED APPLICATION

This application claims priority to German Patent Application No. 10 2015 223 840.7, filed Dec. 1, 2015, which is incorporated herein by reference in its entirety.

### TECHNICAL FIELD

This disclosure relates to hair treatment agents, in particular shampoos and so-called conditioners, containing an active substance combination for gentle and effective hair care.

### BACKGROUND

Care products having an effect that is as long-lasting as possible are becoming increasingly important, not least due to the severe stressing of hair that is caused, for example, by coloring or perming and also by cleaning the hair with shampoos, and by environmental pollution. Such care agents influence the natural structure and the properties of the hair. For instance, the wet and dry combability of the hair and also the hold and body of the hair can be optimized following such care treatments, or the hair can be protected against increased splitting.

It has thus long been customary to subject hair to a special aftertreatment. During this, the hair is treated, for example in the form of conditioning, with specific active substances, for example quaternary ammonium salts or specific polymers. Depending on the formulation, this treatment improves the combability, the hold and the body of the hair and reduces the rate of splitting.

Multifunctional cosmetic products are also known in the prior art. These include in particular the so-called “2 in 1” shampoos, which not only clean but also condition the hair. Such products are greatly appreciated by consumers since, due to the product performance thereof, they enable the omission of at least one method step, for example the conditioning using a conventional hair conditioner.

Products for changing the natural hair color also play a prominent role in hair cosmetics. A distinction is made between permanent, semi-permanent or temporary coloring systems based on chemical and/or natural dyes. However, the hair colors artificially produced by permanent, semi-permanent or temporary coloring systems have the disadvantage that they may change in an undesirable manner, for example during or after hair cleaning.

An “undesirable change” will be understood to mean fading or bleeding or also the loss of color brilliance of the shade achieved by the respective coloring on the hair. Environmental influences and/or the effects of sunlight may further intensify these changes.

The use of divalent metal salts in hair coloring agents to improve the longevity and thus the fastness of the coloring is known from EP 2438900 A1.

There is still a need to provide active substances or active substance combinations for hair treatment agents having good care properties, which moreover strengthen the adhesion of dyes to the hair fibers and thus obtain the fastness of the artificially produced hair color, and to further develop hair treatment agents in this regard.

However, it has been observed that the water hardness can have a negative effect on the care properties of hair treatment agents, so that the care performance of one and the same product is either too low or too high depending on the water quality.

Too high a care performance, or “excessive care” (occurrence of a so-called build-up effect) of the hair, will be understood to mean especially a greasy feel to the hair, little volume to the hair and/or a lank, bedraggled appearance of the hair, whereas damaged hair (brittle, dull hair that is susceptible to splitting) is an indication of too low a care performance.

### BRIEF SUMMARY

Hair treatment agents and methods for treating hair are provided herein. In an embodiment, a hair treatment agent includes:

a) at least one fatty alcohol,

b) at least one cationic surfactant,

c) hexetidine, and

d) phenoxyethanol.

In another embodiment, a method for treating hair includes applying a hair treatment agent to dry or moist hair. The hair treatment agent includes:

a) at least one fatty alcohol,

b) at least one cationic surfactant,

c) hexetidine, and

d) phenoxyethanol.

The hair treatment is agent is left on the hair for a period of 30 to 300 seconds and then rinsed off.

### DETAILED DESCRIPTION

The following detailed description is merely exemplary in nature and is not intended to limit the hair treatment agents and methods for treating hair. Furthermore, there is no intention to be bound by any theory presented in the preceding background or the following detailed description.

The object of the present application was therefore to provide skin-friendly hair treatment agents having a good care effect, which have excellent foaming properties and good rinsability with water and which, in addition, reduce or prevent the washing-out of color from colored hair.

The aim was for the hair treatment agents having a care effect to have a conditioning performance that remains constant regardless of the water quality and in particular not to weigh down fine hair and/or damaged hair and to strengthen the structure of the hair and thus protect it against splitting and breaking and to improve the combability and feel. The aim was also for the agents to have a germ-reducing effect.

It has now been found that a combination of specific ingredients has a particularly positive effect on hair treated therewith and on the hair follicles.

A first subject matter as contemplated herein relates to hair treatment agents containing

a) at least one fatty alcohol,

b) at least one cationic surfactant,

c) hexetidine,

d) phenoxyethanol.

In the context as contemplated herein, hair treatment agents are, for example, hair shampoos, leave-in hair conditioners, conditioning shampoos, hairsprays, rinse-out hair conditioners, intensive hair conditioners, hair masks, hair tonics, perm fixing solutions, hair coloring shampoos, hair coloring agents, hair setting agents, hair smoothing agents, hair styling preparations, blow-drying lotions, mousses, hair gels, hair waxes or combinations thereof. In view of the fact that men in particular are often reluctant to use multiple different agents and/or multiple application steps, preference is given to those agents which a man already uses. Preferred agents are therefore shampoos, conditioners or hair tonics.

The hair treatment agents contain at least one fatty alcohol.

Fatty alcohols are aliphatic, long-chain, monohydric primary alcohols having hydrocarbon radicals which contain 6 to 30, preferably 6 to 22, carbon atoms. The hydrocarbon radicals may be saturated or mono- or polyunsaturated. Fatty alcohols which can be used with preference in the context as contemplated herein are selected from 1-hexanol, 1-heptanol, 1-octanol, 1-decanol, 1-dodecanol (lauryl alcohol), 1-tetradecanol (myristyl alcohol), 1-hexadecanol (cetyl alcohol), 1-heptadecanol (margaryl alcohol), 1-octadecanol (stearyl alcohol), 1-eicosanol (arachidyl alcohol), 1-docosanol (behenyl alcohol), 1-tetracosanol (lignoceryl alcohol), 1-hexacosanol (ceryl alcohol), 1-octacosanol (montanyl alcohol), 1-triacontanol (melissyl alcohol), cis-9-hexadecen-1-ol (palmitoleyl alcohol), cis-9-octadecen-1-ol (oleyl alcohol), trans-9-octadecen-1-ol (elaidyl alcohol), cis-11-octadecen-1-ol, 6,9,12-octadecatrien-1-ol (γ-linolenyl alcohol) and mixtures thereof.

Particularly preferred hair treatment agents are characterized in that they contain, based on their weight, about 0.1 to about 20% by weight, preferably about 0.5 to about 15% by weight, more preferably about 1 to about 10% by weight and in particular about 2 to about 8% by weight fatty alcohol(s) of formula (II)

H₃C—(CH₂)ₖ—CH₂—OH (II),

in which k represents integers from 4 to 28, preferably from 6 to 24, more preferably from 8 to 22 and in particular represents 10, 12, 14, 16, 18 or 20.

Particularly preferred hair treatment agents as contemplated herein contain, based on their weight, about 0.1 to about 20% by weight, preferably about 0.5 to about 15% by weight, more preferably about 1 to about 10% by weight and in particular about 2 to about 8% by weight alcohol(s) from the group consisting of 1-dodecanol (lauryl alcohol), 1-hexadecanol (cetyl alcohol), 1-octadecanol (stearyl alcohol) and 1-docosanol (behenyl alcohol), the amounts relating to the total amount of said fatty alcohols in the composition.

Very particularly preferred hair treatment agents as contemplated herein contain, based on their weight, about 0.1 to about 20% by weight, preferably about 0.5 to about 15% by weight, more preferably about 1 to about 10% by weight and in particular about 2 to about 8% by weight alcohol(s) from the group consisting of 1-hexadecanol (cetyl alcohol) and 1-octadecanol (stearyl alcohol), the amounts relating to the total amount of said fatty alcohols in the composition.

The hair treatment agents contain at least one cationic surfactant.

The cationic surfactant(s) is/are preferably selected from the group consisting of quaternary ammonium compounds and/or amidoamines, preferred cationic surfactant(s) being selected from
alkyltrimethylammonium chlorides having preferably 10 to 18 carbon atoms in the alkyl radical and/or
dialkyldimethylammonium chlorides having preferably 10 to 18 carbon atoms in the alkyl radical and/or
trialkylmethylammonium chlorides having preferably 10 to 18 carbon atoms in the alkyl radical and/or
cetyltrimethylammonium chloride and/or
stearyltrimethylammonium chloride and/or
behenyltrimethylammonium chloride and/or
distearyldimethylammonium chloride and/or
lauryldimethylammonium chloride and/or
lauryldimethylbenzylammonium chloride and/or
tricetylmethylammonium chloride
Quaternium-27 and/or
Quaternium-83.

Hair treatment agents which are preferred as contemplated herein contain, based on their weight, about 0.05 to about 20% by weight, preferably about 0.1 to about 10% by weight, more preferably about 0.25 to about 8% by weight and in particular about 0.5 to about 7% by weight cationic surfactant(s).

Particularly preferred cationic surfactants are selected from compounds of the following formula (I)

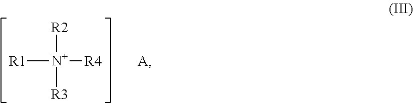

in which at most three radicals R1 to R4 independently of one another represent a saturated or unsaturated, branched or unbranched alkyl group having 1 to 4 C atoms, at least one radical R1 to R4 represents a saturated or unsaturated, branched or unbranched alkyl chain having 8 to 30 C atoms, and A denotes a physiologically acceptable organic or inorganic anion.

In preferred compounds of formula (I),
two or three radicals R1 to R4 represent a methyl or ethyl group,
one or two radical(s) R1 to R4 represent(s) a saturated or unsaturated, branched or unbranched alkyl chain having 14 to 26 C atoms, and
A represents a halide ion, a sulfate ion of general formula RSO₃⁻, in which R has the meaning of saturated or unsaturated alkyl radicals having 1 to 4 carbon atoms, or represents an anionic radical of an organic acid such as maleic acid, fumaric acid, oxalic acid, tartaric acid, citric acid, lactic acid or acetic acid.

More preference is given to compounds of formula (III) in which
three radicals R1 to R4 represent a methyl group,
one radical R1 to R4 represents a cetyl, palmityl, stearyl, arachidyl or behenyl group, and
A represents a chloride or a methosulfate ion.

With particular preference, the at least one compound of formula (I) is selected from cetyltrimethylammonium chloride, cetyltrimethylammonium methosulfate, behentrimethylammonium chloride and/or behentrimethylammonium methosulfate. These compounds may be used individually or in combination in the agent as contemplated herein, the total amount of compounds of formula (I) in the agent preferably being at most about 10% by weight, and the stated amounts relating to the total weight of the agent as contemplated herein.

In particular, an agent as contemplated herein preferably contains behentrimethylammonium chloride as the cationic surfactant. Preference is given here to hair treatment agents as contemplated herein which contain, based on their weight, about 0.05 to about 20% by weight, preferably about 0.1 to about 10% by weight, more preferably about 0.25 to about 8% by weight and in particular about 0.5 to about 7% by weight behenyltrimethylammonium chloride.

The hair treatment agents may also contain at least one esterquat as cationic surfactant.

In the context as contemplated herein, “esterquats” will preferably be understood to mean compounds of the following formula (IV)

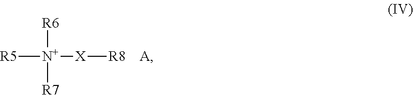

in which
the radicals R5, R6 and R7 each independently of one another may be identical or different and have the following meaning:
a saturated or unsaturated, branched or unbranched alkyl radical having 1 to 4 carbon atoms, which may contain at least one hydroxyl group, or
a saturated or unsaturated, branched or unbranched or a cyclic saturated or unsaturated alkyl radical having 6 to 30 carbon atoms, which may contain at least one hydroxyl group, or
an aryl or alkylaryl radical, for example phenyl or benzyl, or
(—X—R8), with the proviso that at most 2 of the radicals R5, R6 or R7 can represent (—X—R8), wherein
   X has the following meaning:
—(CH2)n- where n=1 to 20, preferably n=1 to 10 and particularly preferably n=1 to 5, or
—(CH₂—CHR9-O)ₙ— where n=1 to 200, preferably 1 to 100, more preferably 1 to 50, and particularly preferably 1 to 20, and where R9 denotes hydrogen, methyl or ethyl, or
a hydroxyalkylene group having one to four carbon atoms, which may be branched or unbranched, and which contains at least one and at most 3 hydroxyl groups, and wherein
   R8 has the following meaning:
R10-O—CO—, in which R10 is a saturated or unsaturated, branched or unbranched or a cyclic, saturated or unsaturated alkyl radical having 6 to 30 carbon atoms, which may contain at least one hydroxyl group, and which may optionally also be ethoxylated with 1 to 100 ethylene oxide units and/or 1 to 100 propylene oxide units, or
R11-CO—, in which R11 is a saturated or unsaturated, branched or unbranched or a cyclic, saturated or unsaturated alkyl radical having 6 to 30 carbon atoms, which may contain at least one hydroxyl group, and which may optionally also be ethoxylated with 1 to 100 ethylene oxide units and/or 1 to 100 propylene oxide units, and
   in which A represents a physiologically acceptable organic or inorganic anion,
   preferably one of the radicals R5, R6 or R7 represents the group (—X—R8), R8 represents a non-ethoxylated fatty acid residue, such as a palmitic, stearic, arachidic or behenic acid residue, in particular a stearic acid residue, and A represents a halide ion, a sulfate ion of general formula RSO₃⁻, in which R has the meaning of saturated or unsaturated alkyl radicals having 1 to 4 carbon atoms, or an anionic residue of an organic acid such as maleic acid, fumaric acid, oxalic acid, tartaric acid, citric acid, lactic acid or acetic acid, in particular a chloride ion or a methosulfate ion.

The esterquats suitable for the agents as contemplated herein are preferably selected from at least one of the products marketed under the trade names Rewoquat®, Stepantex®, Dehyquart®, Armocare® and Akypoquat®. Specific examples of esterquats which are particularly suitable as contemplated herein are the products Armocare® VGH-70, Dehyquart° F.-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131.

Particular preference is given to agents as contemplated herein which contain as the esterquat at least one of the compounds known under the INCI names Distearoylethyl Hydroxyethylmonium Methosulfate and Distearoylethyl Hydroxyethylmonium Chloride.

Preference is given in particular to Distearoylethyl Hydroxyethylmonium Methosulfate, which may be contained in the agents as contemplated herein in a preferred amount of about 0.1 to about 10% by weight, more preferably about 0.5 to about 8% by weight, particularly preferably about 0.75 to about 6% by weight and in particular about 1 to about 5% by weight, the stated amounts relating to the total weight of the agent as contemplated herein.

The esterquat(s) may be added to the agents as contemplated herein either individually or as a mixture with other active care substances.

For reasons of better handling and processability, it may be advantageous if the esterquat(s), in particular Distearoylethyl Hydroxyethylmonium Methosulfate, is (are) added to the agents as contemplated herein as an active substance mixture. One particularly suitable example of such an active substance mixture is available, for example, under the trade name Dehyquart® F 75 from the company BASF (Distearoylethyl Hydroxyethylmonium Methosulfate and Cetearyl Alcohol).

The hair treatment agents contain hexetidine. Hexetidine, also called 5-amino-1,3-bis(2-ethylhexal)hexahydro-5-methylpyrimidine or 1,3-bis(2-ethylhexyl)hexahydro-5-methylpyrimidin-5-amine is an antiseptic or disinfectant which is represented by the following formula:

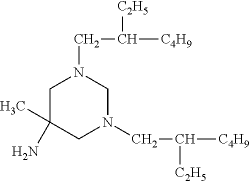

Hexetidine is used in the agents as contemplated herein preferably within specific relatively narrow amount ranges. Hair treatment agents which are preferred as contemplated herein contain, based on their weight, about 0.001 to about 0.3% by weight, preferably about 0.01 to about 0.2% by weight, more preferably about 0.025 to about 0.15% by weight and in particular about 0.05 to about 0.1% by weight hexetidine.

The hair treatment agents contain phenoxyethanol. Phenoxyethanol is also used in the agents as contemplated herein preferably within specific relatively narrow amount ranges. Hair treatment agents which are preferred as contemplated herein contain, based on their weight, about 0.01 to about 5% by weight, preferably about 0.025 to about 3% by weight, more preferably about 0.05 to about 2% by weight and in particular about 0.1 to about 1% by weight phenoxyethanol.

The hair treatment agents may contain at least one cationic polysaccharide polymer.

Cationic polysaccharide polymers increase the care performance of the hair treatment agents as contemplated herein (in particular the efficacy of the agents as contemplated herein against hair breakage). Suitable cationic polysaccharide polymers may be selected from cationic cellulose compounds and/or cationic guar derivatives.

Particularly preferred hair treatment agents as contemplated herein contain as cationic polysaccharide polymer(s), based on the weight of the agent, about 0.01 to about 3% by weight, preferably about 0.05 to about 2% by weight, more preferably about 0.1 to about 1.5% by weight and in particular about 0.15 to about 0.8% by weight of at least one polymer from the group consisting of cationic cellulose polymers and/or cationic guar derivatives.

In the context as contemplated herein, cationic cellulose compounds are those which carry more than one permanent cationic charge in at least one side chain. Cellulose is constructed from beta-1,4-glycoside-linked D-glucopyranose units and forms unbranched, water-insoluble chains. The term “side chain” of a cellulose defines chemical substituents which bind to the cellulose backbone and are not part of the native cellulose, since they have been introduced subsequently, for example, by chemical synthesis.

Preference is given to quaternized cellulose polymers derived from hydroxy(C₂-C₄)alkylcelluloses, particularly preferably from hydroxyethylcelluloses.

Such polymers are known to the person skilled in the art and are commercially available from various companies. Particular preference is given to the cationic cellulose derivatives known under the INCI names Polyquaternium-4, Polyquaternium-10, Polyquaternium-24, Polyquaternium-67 and/or Polyquaternium-72. Very particular preference is given to Polyquaternium-10, Polyquaternium-24 and/or Polyquaternium-67, preference being given in particular to Polyquaternium-10.

Preferred hair treatment agents as contemplated herein contain as cationic polysaccharide polymer(s), based on the weight of the agent, about 0.01 to about 3% by weight, preferably about 0.05 to about 2% by weight, more preferably about 0.1 to about 1.5% by weight and in particular about 0.15 to about 0.8% by weight of at least one polymer from the group consisting of Polyquaternium-4, Polyquaternium-10, Polyquaternium-24, Polyquaternium-67 and/or Polyquaternium-72.

Particularly preferred hair treatment agents as contemplated herein contain as cationic polysaccharide polymer(s), based on the weight of the agent, about 0.01 to about 3% by weight, preferably about 0.05 to about 2% by weight, more preferably about 0.1 to about 1.5% by weight and in particular about 0.15 to about 0.8% by weight Polyquaternium-10.

Suitable cationic guar derivatives in the context of the invention are cationic hydroxyalkyl guar derivatives, preferably cationic hydroxyethyltrimethylammonium guar and/or cationic hydroxypropyltrimethylammonium guar having average molecular weights of between about 100,000 and about 2,000,000 daltons. Particular preference is given to the cationic guar polymers known under the INCI name Guar Hydroxypropyltrimonium Chloride, having a molecular weight (weight-average) of between about 200,000 and about 1,600,000 daltons. The cationic charge density of said guar polymers is preferably at least 0.4 meq/g, more preferably at least 0.5 meq/g and in particular at least 0.6 meq/g. The nitrogen content thereof preferably lies in the range from about 1.1 to about 1.8% by weight (based on their total weight).

Cationic guar derivatives which are known under the INCI name Guar Hydroxypropyltrimonium Chloride are known to the person skilled in the art and are available from various suppliers, for example, under the trade names Cosmedia® Guar, N-Hance® and/or Jaguar®.

Particularly preferred hair treatment agents as contemplated herein contain as cationic polysaccharide polymer(s), based on the weight of the agent, about 0.01 to about 3% by weight, preferably about 0.05 to about 2% by weight, more preferably about 0.1 to about 1.5% by weight and in particular about 0.15 to about 0.8% by weight Guar Hydroxypropyltrimonium Chloride.

The hair treatment agents may contain (an) organic acid(s).

Typical representatives of organic acids are aliphatic mono- and dicarboxylic acids such as, for example, acetic acid, propionic acid, oxalic acid and 1,3-propanedioic acid, as well as aromatic carboxylic acids such as, for example, benzoic acid. Further organic acids are, for example, hydroxycarboxylic acids such as glycolic acid, citric acid, tartaric acid, malic acid and lactic acid. Unsaturated mono- or dicarboxylic acids such as, for example, fumaric acid or α-ketocarboxylic acids such as, for example, pyruvic acid (2-oxopropionic acid) are also contemplated herein.

Hair treatment agents which are preferred as contemplated herein contain, based on their weight, about 0.001 to about 10% by weight, preferably about 0.01 to about 5% by weight, more preferably about 0.05 to about 3% by weight and in particular about 0.1 to about 2% by weight organic acid(s).

Particularly preferred hair treatment agents contain, based on their weight, about 0.001 to about 10% by weight, preferably about 0.01 to about 5% by weight, more preferably about 0.05 to about 3% by weight and in particular about 0.1 to about 2% by weight organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-napthalenepentaacetic acid, malonaldehydic acid, 4-hydroxyphthalamidic acid, 1-pyrazolecarboxylic acid, gallic acid or propanetricarboxylic acid, glycolic acid, lactic acid, malic acid, tartaric acid or citric acid.

Among the aforementioned acids, some representatives are particularly preferred because they additionally give the hair treated with the agents as contemplated herein a pleasant shine. Extremely preferred hair treatment agents contain, based on their weight, about 0.001 to about 10% by weight, preferably about 0.01 to about 5% by weight, more preferably about 0.05 to about 3% by weight and in particular about 0.1 to about 2% by weight organic acid(s) from the group consisting of formic acid, oxalic acid, maleic acid, lactic acid, tartaric acid or citric acid.

The hair treatment agents contain the above-described active substances preferably in a cosmetically acceptable carrier. In the context as contemplated herein, this will be understood to mean preferably an aqueous or aqueous-alcoholic carrier.

The cosmetic carrier contains preferably at least about 50% by weight, more preferably at least about 60% by weight, particularly preferably at least about 70% by weight and more particularly preferably at least about 75% by weight water.

The cosmetic carrier may also contain about 0.01 to about 40% by weight, preferably about 0.05 to about 30% by weight and in particular about 0.1 to about 20% by weight of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol or mixtures of said alcohols.

The water-soluble alcohols are particularly preferred. Particular preference is given to ethanol, 1,2-propylene glycol, glycerol, benzyl alcohol and mixtures of said alcohols.

For very good (scalp) skin tolerability of the hair treatment agents as contemplated herein, it is advantageous if said agents have a slightly acidic pH value.

It has been found that the agents as contemplated herein are particularly well tolerated by the skin and are particularly gentle in a pH range of from 4.2 to 5.8.

In a first preferred embodiment, the hair treatment agents as contemplated herein therefore preferably have a pH in the range from 4.2 to 5.8, more preferably from 4.25 to 5.6, particularly preferably from 4.3 to 5.5, extremely preferably from 4.35 to 5.4 and more particularly preferably from 4.4 to 5.3.

The hair treatment agents as contemplated herein may contain plant oils, plant butters and/or plant waxes. These plant oil components give the hair improved combability and manageability and increase the shine of the hair.

Suitable plant oil components include natural (plant) oils and/or butters, which usually contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheatgerm oil, pumpkin seed oil, mallow oil, hazelnut oil, safflower oil, canola oil, sasanqua oil, jojoba oil, rambutan oil, cocoa butter and/or shea butter.

As suitable natural or plant waxes, use may preferably be made of carnauba wax, beeswax and/or candelilla wax.

Particularly preferred plant oil components are (sweet) almond oil, peach kernel oil, apricot kernel oil, amaranth seed oil, argan oil, olive oil, jojoba oil, cocoa butter and/or shea butter.

Apricot kernel oil, argan oil, olive oil and/or jojoba oil are more particularly preferred.

In one preferred embodiment, the hair treatment agents as contemplated herein preferably contain coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheatgerm oil, pumpkin seed oil, mallow oil, hazelnut oil, safflower oil, canola oil, sasanqua oil, jojoba oil, rambutan oil, cocoa butter and/or shea butter.

Within this embodiment, it is particularly preferred if the hair treatment agents as contemplated herein contain (sweet) almond oil, peach kernel oil, apricot kernel oil, amaranth seed oil, argan oil, olive oil, jojoba oil, cocoa butter and/or shea butter.

The proportion by weight of the at least one plant oil, of the plant butter and/or of the plant wax relative to the total weight of the hair treatment agents as contemplated herein is preferably about 0.02 to about 2.50% by weight, more preferably about 0.03 to about 2.00% by weight, particularly preferably about 0.04 to about 1.50% by weight and in particular about 0.05 to about 1.00% by weight.

Besides the aforementioned essential and optional constituents, the hair treatment agents as contemplated herein may in another preferred embodiment contain, in order to further increase the care properties of the agents, at least one further hair-conditioning active substance which may be selected from the group consisting of
protein hydrolysates,
vitamins,
plant extracts and/or
glycerol.

Suitable protein hydrolysates are to be understood to mean product mixtures which can be obtained by acidically, basically or enzymatically catalyzed breakdown of proteins.

Protein hydrolysates of plant, animal and/or marine origin may be used.

Animal protein hydrolysates are, for example, elastin, collagen, keratin, silk and milk protein hydrolysates, which may also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Preference is given to protein hydrolysates of plant origin, for example soy, almond, rice, pea, potato and wheat protein hydrolysates. Such products are available, for example, under the trademarks Gluadin® (Cognis), DiaMin® (Diamalt), Lexein® (Inolex) and Crotein® (Croda). Cationized protein hydrolysates can also be used, wherein the underlying protein hydrolysate can derive from animal sources, for example from collagen, milk or keratin, from plant sources, for example from wheat, maize, rice, potatoes, soy or almonds, from marine life forms, for example from fish collagen or algae, or from protein hydrolysates obtained by biotechnology. The protein hydrolysates underlying the cationic derivatives can be obtained from the corresponding proteins by means of a chemical, in particular alkaline or acid hydrolysis, an enzymatic hydrolysis and/or a combination of both types of hydrolysis. The hydrolysis of proteins generally gives rise to a protein hydrolysate having a molecular weight distribution from approximately about 100 daltons to up to several thousand daltons. Preference is given to those cationic protein hydrolysates with an underlying protein component which has a molecular weight of from about 100 to about 25,000 daltons, preferably about 250 to about 5000 daltons. Cationic protein hydrolysates are also to be understood to include quaternized amino acids and mixtures thereof. The quaternization of the protein hydrolysates or amino acids is frequently performed using quaternary ammonium salts such as, for example, N,N-dimethyl-N-(n-alkyl)-N-(2-hydroxy-3-chloro-n-propyl) ammonium halides. The cationic protein hydrolysates can moreover also be further derivatized. Typical examples of cationic protein hydrolysates and derivatives are the commercially available products known under the following INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The proportion by weight of the protein hydrolysate(s) relative to the total weight of the hair treatment agents is preferably about 0.01 to about 5% by weight, more preferably about 0.025 to about 3% by weight and in particular about 0.05 to about 2% by weight.

Regardless of the source (plant, animal, marine, etc.), protein hydrolysates contain individual amino acids, oligopeptides and optionally polypeptides, depending on the degree of hydrolysis.

With particular preference, the hair treatment agents as contemplated herein contain at least one oligopeptide which has at least one amino acid sequence Glu-Glu-Glu

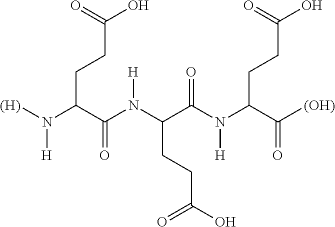

wherein the amino group may be present in free or protonated form and the carboxy groups may be present in free or deprotonated form.

Preferred hair treatment agents as contemplated herein are characterized in that they contain, based on their weight, about 0.0001 to about 10% by weight of at least one oligopeptide which has at least one amino acid sequence Glu-Glu-Glu

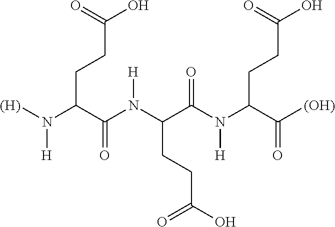

wherein the amino group may be present in free or protonated form and the carboxy groups may be present in free or deprotonated form.

In this, as in all of the formulae below, the bracketed hydrogen atom of the amino group, like the bracketed hydroxyl group of the acid function, means that the groups in question may be present as such (in which case it is an oligopeptide having the respective number of amino acids as shown (in formula 3 above)) or that the amino acid sequence is present in an oligopeptide which also comprises further amino acids. Depending on where the further amino acid(s) is/are bound, the bracketed constituents in the above formula are replaced by the other amino acid residue(s).

These preferred hair treatment agents as contemplated herein contain, based on their weight, about 0.0001 to about 10% by weight of at least one oligopeptide which has at least one amino acid sequence Glu-Glu-Glu, that is to say at least three successive glutamic acids.

In the context of the present application, oligopeptides are amino acid condensation products which are linked in an acid amide-like manner by peptide bonds and which comprise at least 3 and at most 25 amino acids.

In preferred hair treatment agents as contemplated herein, the oligopeptide comprises 5 to 15 amino acids, preferably 6 to 13 amino acids, particularly preferably 7 to 12 amino acids and in particular 8, 9 or 10 amino acids.

The molar mass of the oligopeptide contained in the agents as contemplated herein may vary depending on whether further amino acids are bound to the Glu-Glu-Glu sequence and depending on the type of said amino acids. Preferred hair treatment agents which are used as contemplated herein are characterized in that the oligopeptide has a molecular weight of about 650 to about 3000 Da, preferably about 750 to about 2500 Da, particularly preferably about 850 to about 2000 Da and in particular about 1000 to about 1600 Da.

To sum up, preferred hair treatment agents are characterized in that the oligopeptide comprises 5 to 15 amino acids, preferably 6 to 13 amino acids, particularly preferably 7 to 12 amino acids and in particular 8, 9 or 10 amino acids and has a molecular weight of about 650 to about 3000 Da, preferably about 750 to about 2500 Da, particularly preferably about 850 to about 2000 Da and in particular about 1000 to about 1600 Da.

As can be seen from the preferred number of amino acids in the oligopeptides and the preferred molecular weight range, use is preferably made of oligopeptides which do not consist solely of the three glutamic acids but rather have further amino acids bound to said sequence. These further amino acids are preferably selected from specific amino acids, while specific other representatives are less preferred as contemplated herein.

For instance, it is preferred if the oligopeptides used in the agents as contemplated herein contain no methionine.

It is also preferred if the oligopeptides used in the agents as contemplated herein contain no cysteine and/or cystine.

It is also preferred if the oligopeptides used in the agents as contemplated herein contain no aspartic acid and/or asparagine.

It is also preferred if the oligopeptides used in the agents as contemplated herein contain no serine and/or threonine.

In contrast, it is preferred if the oligopeptides used in the agents as contemplated herein contain tyrosine.

It is also preferred if the oligopeptides used in the agents as contemplated herein contain leucine.

It is also preferred if the oligopeptides used in the agents as contemplated herein contain isoleucine.

It is also preferred if the oligopeptides used in the agents as contemplated herein contain arginine.

It is also preferred if the oligopeptides used in the agents as contemplated herein contain valine.

Particularly preferred oligopeptides and amino acid sequences contained in the preferred oligopeptides will be described below:

One particularly preferred oligopeptide additionally contains tyrosine, which is preferably bound via its acid function to the Glu-Glu-Glu sequence. Hair treatment agents which are preferred as contemplated herein are therefore characterized in that the oligopeptide contained therein has at least one amino acid sequence Tyr-Glu-Glu-Glu

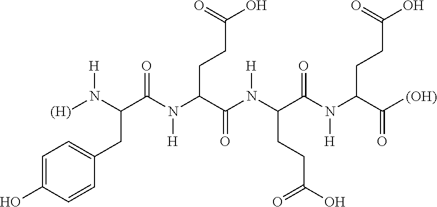

wherein the amino group may be present in free or protonated form and the carboxy groups may be present in free or deprotonated form.

Another particularly preferred oligopeptide additionally contains isoleucine, which is preferably bound via its amino function to the Glu-Glu-Glu sequence. Hair treatment agents which are preferred as contemplated herein are therefore characterized in that the oligopeptide contained therein has at least one amino acid sequence Glu-Glu-Glu-Ile

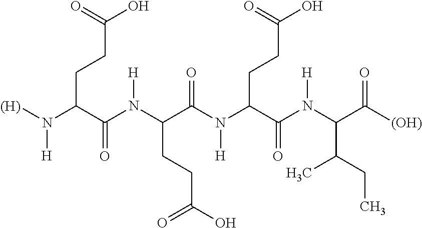

wherein the amino group may be present in free or protonated form and the carboxy groups may be present in free or deprotonated form.

As contemplated herein, preference is given to oligopeptides which contain both of the aforementioned amino acids (tyrosine and isoleucine). Particular preference is given to hair treatment agents as contemplated herein in which the oligopeptide contained in the hair treatment agent has at least one amino acid sequence Tyr-Glu-Glu-Glu-Ile

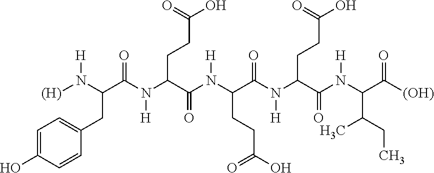

wherein the amino group may be present in free or protonated form and the carboxy groups may be present in free or deprotonated form.

Further preferred oligopeptides additionally contain arginine, which is preferably bound to isoleucine.

Particular preference is given to hair treatment agents as contemplated herein in which the oligopeptide contained in the hair treatment agent has at least one amino acid sequence Tyr-Glu-Glu-Glu-Ile-Arg

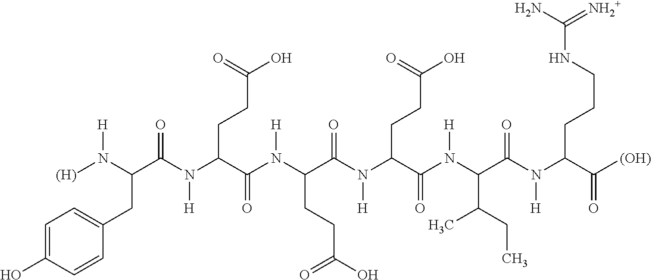

wherein the amino groups may be present in free or protonated form and the carboxy groups may be present in free or deprotonated form.

Yet further preferred oligopeptides additionally contain valine, which is preferably bound to the arginine. Hair treatment agents which are further preferred as contemplated herein are therefore characterized in that the oligopeptide contained in the hair treatment agent has at least one amino acid sequence Tyr-Glu-Glu-Glu-Ile-Arg-Val

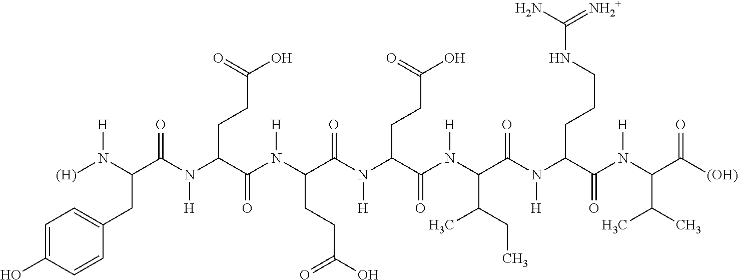

wherein the amino groups may be present in free or protonated form and the carboxy groups may be present in free or deprotonated form.

Yet further preferred oligopeptides additionally contain leucine, which is preferably bound to the valine. Hair treatment agents which are further preferred as contemplated herein are therefore characterized in that the oligopeptide contained in the hair treatment agent has at least one amino acid sequence Tyr-Glu-Glu-Glu-Ile-Arg-Val-Leu

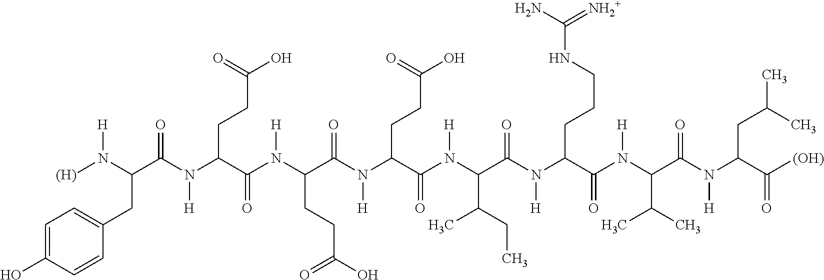

wherein the amino groups may be present in free or protonated form and the carboxy groups may be present in free or deprotonated form.

Particularly preferred oligopeptides additionally contain leucine, which is preferably bound to the tyrosine. Hair treatment agents which are further preferred as contemplated herein are therefore characterized in that the oligopeptide contained in the hair treatment agent has at least one amino acid sequence Leu-Tyr-Glu-Glu-Glu-Ile-Arg-Val-Leu

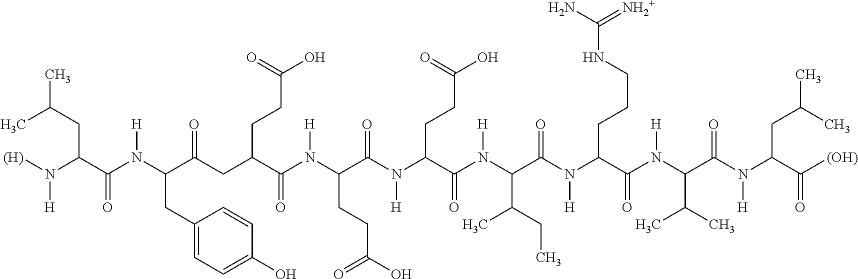

wherein the amino groups may be present in free or protonated form and the carboxy groups may be present in free or deprotonated form.

With very particular preference, agents as contemplated herein contain at least two oligopeptides which satisfy the aforementioned criteria but differ from one another. For example, preference is given to the use of hair treatment agents which contain at least two mutually different oligopeptides A and B which both contain the amino acid sequence Glu-Glu-Glu.

Such mutually different oligopeptides A and B correspond to one another in that they carry three successive Glu amino acids in their amino acid sequence but exhibit differences in the amino acids bound in front of or behind said Glu amino acids. Preference is given to mutually different peptides with partial correspondence, which correspondence may be much greater than in the aforementioned three amino acids.

Further preferred hair treatment agents are thus characterized in that the hair treatment agent contains at least two mutually different oligopeptides A and B which both contain the amino acid sequence Glu-Glu-Glu-Ile.

Preference is also given to hair treatment agents which contain at least two mutually different oligopeptides A and B which both contain the amino acid sequence Tyr-Glu-Glu-Glu.

Yet further preferred hair treatment agents are characterized in that they contain at least two mutually different oligopeptides A and B which both contain the amino acid sequence Glu-Glu-Glu-Ile-Arg.

Likewise yet further preferred hair treatment agents are characterized in that the hair treatment agent contains at least two mutually different oligopeptides A and B which both contain the amino acid sequence Tyr-Glu-Glu-Glu-Ile.

Preferred hair treatment agents as contemplated herein are therefore characterized in that the oligopeptide has at least one amino acid sequence Tyr-Glu-Glu-Glu-Ile

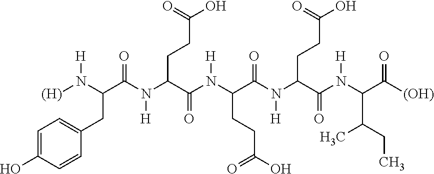

wherein the amino group may be present in free or protonated form and the carboxy groups may be present in free or deprotonated form.

Very particularly preferred hair treatment agents are characterized in that the hair treatment agent contains at least two mutually different oligopeptides A and B which both contain the amino acid sequence Glu-Glu-Glu-Ile-Arg.

Likewise very particularly preferred hair treatment agents are characterized in that the hair treatment agent contains at least two mutually different oligopeptides A and B which both contain the amino acid sequence Tyr-Glu-Glu-Glu-Ile-Arg.

There is preferably an even greater structural correspondence in the oligopeptides. For instance, hair treatment agents containing at least two mutually different oligopeptides A and B which both contain the amino acid sequence Glu-Glu-Glu-Ile-Arg-Val are further preferred embodiments as contemplated herein.

Hair treatment agents containing at least two mutually different oligopeptides A and B which both contain the amino acid sequence Tyr-Glu-Glu-Glu-Ile-Arg-Val are likewise preferred embodiments.

Yet further preferred hair treatment agents as contemplated herein are characterized in that they contain at least two mutually different oligopeptides A and B which both contain the amino acid sequence Glu-Glu-Glu-Ile-Arg-Val-Leu.

Likewise yet further preferred hair treatment agents as contemplated herein are characterized in that they contain at least two mutually different oligopeptides A and B which both contain the amino acid sequence Tyr-Glu-Glu-Glu-Ile-Arg-Val-Leu.

Preferred hair treatment agents as contemplated herein are therefore characterized in that the oligopeptide has at least one amino acid sequence Tyr-Glu-Glu-Glu-Ile-Arg-Val-Leu

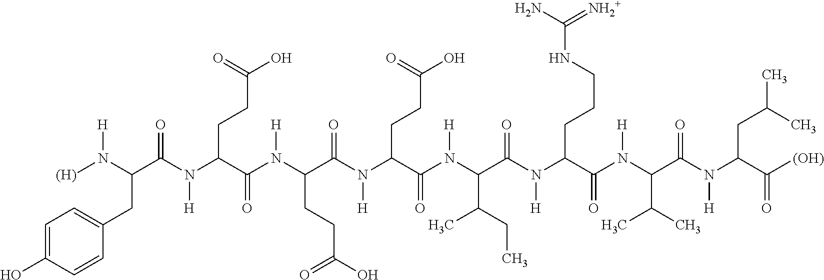

wherein the amino groups may be present in free or protonated form and the carboxy groups may be present in free or deprotonated form.

Particularly preferred hair treatment agents as contemplated herein are characterized in that they contain at least two mutually different oligopeptides A and B, wherein the oligopeptide A has the amino acid sequence Leu-Tyr-Glu-Glu-Glu-Ile-Arg-Val-Leu

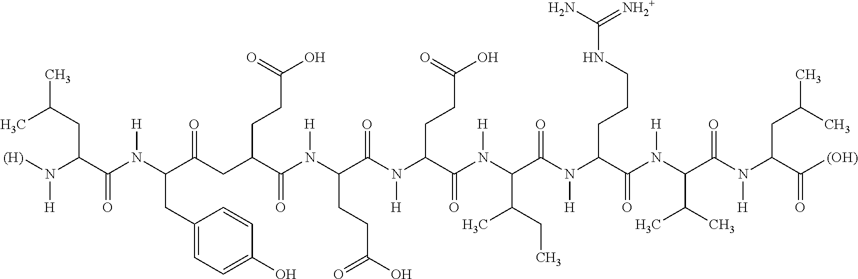

wherein the amino groups may be present in free or protonated form and the carboxy groups may be present in free or deprotonated form
and the oligopeptide B has the amino acid sequence Tyr-Glu-Glu-Glu-Ile-Arg-Val-Leu

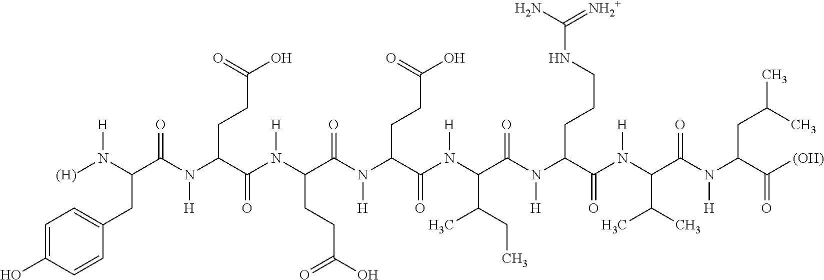

wherein the amino groups may be present in free or protonated form and the carboxy groups may be present in free or deprotonated form.

In very particularly preferred hair treatment agents of this last-mentioned embodiment, said agents contain, based on the weight of the agent, about 0.00001 to about 1% by weight oligopeptide A and about 0.00001 to about 1% by weight oligopeptide B.

In further preferred hair treatment agents of this last-mentioned embodiment, said agents contain, based on the weight of the agent, about 0.00005 to about 0.1% by weight oligopeptide A and about 0.00005 to about 0.1% by weight oligopeptide B.

In yet further preferred hair treatment agents of this last-mentioned embodiment, said agents contain, based on the weight of the agent, about 0.0001 to about 0.01% by weight oligopeptide A and about 0.0001 to about 0.001% by weight oligopeptide B.

The oligopeptides used in the context as contemplated herein, which satisfy the conditions mentioned above, can advantageously be obtained from keratin materials. It is preferred as contemplated herein that said oligopeptides are used in high proportions relative to the total keratin peptide content of the agents.

It is very particularly preferred that a highest possible proportion of all the keratin peptides contained in the agent as contemplated herein satisfies the conditions mentioned above.

Preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all keratin peptides contained in the agent have the amino acid sequence Glu-Glu-Glu.

Further preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Glu-Glu-Glu-Ile.

Yet further preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu.

Particularly preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile.

Very particularly preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile-Arg.

Yet further preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile-Arg-Val.

Particularly preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile-Arg-Val-Leu.

The conditions mentioned above relate to the total content, in the agents as contemplated herein, of peptides originating from keratin materials. In addition to the oligopeptides of keratin origin, use may of course be made of further peptides and/or protein hydrolysates, for example from other native sources. Preference is given, for example, to the additional use of wheat protein hydrolysates.

Suitable vitamins are to be understood to mean preferably the following vitamins, provitamins and vitamin precursors, and derivatives thereof:
Vitamin A: The group of substances designated as vitamin A includes retinol (vitamin A₁) and also 3,4-didehydroretinol (vitamin A₂). β-Carotene is the provitamin of retinol. As vitamin A components, mention may be made of, for example, vitamin A acid and esters thereof, vitamin A aldehyde and vitamin A alcohol and esters thereof such as the palmitate and acetate.
Vitamin B: The vitamin B group or the vitamin B complex includes inter alia
   Vitamin B₁ (thiamine)
   Vitamin B₂ (riboflavin)
   Vitamin B₃. The compounds nicotinic acid and nicotinamide (niacinamide) are often included under this designation.
   Vitamin B₅ (pantothenic acid and panthenol). In the context of this group, use is preferably made of panthenol. Derivatives of panthenol which can be used are in particular the esters and ethers of panthenol, pantolactone and also cationically derivatized panthenols. Individual representatives are, for example, panthenol triacetate, panthenol monoethyl ether and the monoacetate thereof, as well as cationic panthenol derivatives.
   Vitamin B₆ (pyridoxine as well as pyridoxamine and pyridoxal).
Vitamin C (ascorbic acid). Use in the form of the palmitate ester, the glucosides or phosphates may be preferred. Use in combination with tocopherols may likewise be preferred.
Vitamin E (tocopherols, in particular α-tocopherol).
Vitamin F: The term “vitamin F” is usually understood to mean essential fatty acids, in particular linoleic acid, linolenic acid and arachidonic acid.
Vitamin H: The compound (3aS,4S,6aR)-2-oxohexahydrothienol[3,4-d]imidazole-4-valeric acid is designated as vitamin H, for which the trivial name biotin has become accepted.

Particular preference is given to vitamins, provitamins and vitamin precursors from groups A, B, E and H. Nicotinamide, biotin, pantolactone and/or panthenol are preferred in particular.

The proportion by weight of the vitamin(s), vitamin derivative(s) and/or vitamin precursor(s) relative to the total weight of the hair treatment agents is preferably about 0.001 to about 2% by weight, particularly preferably about 0.005 to about 1% by weight and in particular about 0.01 to about 0.5% by weight.

Suitable plant extracts are to be understood to mean extracts which can be produced from all parts of a plant. These extracts are usually produced by extraction of the entire plant. In individual cases, however, it may also be preferred to produce the extracts exclusively from blossoms and/or leaves of the plant. The extracts from green tea, oak bark, stinging nettle, witch hazel, hops, chamomile, burdock root, horsetail, whitethorn, lime blossom, lychee, almond, aloe vera, pine needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, lemon balm, restharrow, coltsfoot, marshmallow, ginseng, ginger root, Echinacea purpurea, Olea europaea, Boerhavia diffusa roots, Foeniculum vulgaris and Apium graveolens are especially suitable.

The extracts from green tea, stinging nettle, witch hazel, chamomile, aloe vera, ginseng, Echinacea purpurea, Olea europea and/or Boerhavia diffusa roots are particularly preferred for use in the compositions as contemplated herein.

As extraction agents for producing the aforementioned plant extracts, use may be made of water, alcohols and mixtures thereof. Among the alcohols, preference is given to lower alcohols such as ethanol and isopropanol, but in particular polyhydric alcohols such as ethylene glycol and propylene glycol, both as the sole extraction agent and in a mixture with water. Plant extracts based on water/propylene glycol in a ratio of 1:10 to 10:1 have proven to be particularly suitable.

The plant extracts may be used either in pure form or in dilute form. When used in dilute form, they usually contain approximately about 2 to about 80% by weight active substance, and as the solvent the extraction agent or extraction agent mixture used for the production of said plant extracts.

The plant extracts can be used in the hair treatment agents as contemplated herein (based on the total weight of the agent) preferably in an amount of about 0.01 to about 10% by weight, more preferably about 0.05 to about 7.5% by weight and in particular about 0.1 to about 5% by weight.

Glycerol may be added separately to the hair cleaning and care agents in an amount of up to about 10% by weight (based on the total weight of the agent). However, it may also be a constituent of the aforementioned aqueous-alcoholic carrier.

It has been found that the hair treatment agents as contemplated herein are also suitable for use as an antidandruff preparation.

The total weight of antidandruff agents relative to the total weight of the hair treatment agents may be preferably about 0.01 to about 10% by weight, more preferably about 0.025 to about 7.5% by weight, particularly preferably about 0.05 to about 5% by weight and in particular about 0.075 to about 3% by weight.

Suitable antidandruff active substances may be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts.

Preference is given to climbazole, zinc pyrithione and piroctone olamine.

Further active substances, auxiliaries and additives which may preferably be contained in the hair treatment agents as contemplated herein are, for example:
humectants,
perfumes,
UV filters,
thickeners such as gelatins or plant gums, for example agar-agar, guar gum, alginates, xanthan gum, gum arabic, karaya gum, locust bean gum, flaxseed gums, dextrans, cellulose derivatives, for example methylcellulose, hydroxyalkylcellulose and carboxymethylcellulose, starch fractions and derivatives such as amylose, amylopectin and dextrins, clays and phyllosilicates such as, for example, bentonite, or fully synthetic hydrocolloids such as, for example, polyvinyl alcohol, Ca, Mg or Zn soaps,
structurants such as maleic acid and lactic acid,
dimethyl isosorbide,
cyclodextrins,
active substances for improving the fiber structure, in particular mono-, di- and oligosaccharides such as, for example, glucose, galactose, fructose, fruit sugars and lactose,
colorants for coloring the agent,
active substances such as bisabolol and/or allantoin,
complexing agents such as EDTA, NTA, β-alaninediacetic acid and phosphonic acids,
ceramides. Ceramides are understood to be N-acylsphingosine (fatty acid amides of sphingosine) or synthetic analogs of such lipids (so-called pseudo-ceramides),
propellants such as propane-butane mixtures, N₂O, dimethyl ether, CO₂ and air,
antioxidants,
additional viscosity regulators such as salts (NaCl).

The agents as contemplated herein are preferably so-called rinse-off products, that is to say they are rinsed out of the hair again after a given leave-in time. This leave-in time is preferably less than one hour, that is to say the user preferably does not leave the products in the hair until the next time the hair is washed.

Another subject matter as contemplated herein is therefore a method for treating hair, in which an agent as contemplated herein is applied to dry or wet hair, is left thereon for a duration of from about 30 to about 300 seconds, and then is rinsed out.

The agents as contemplated herein lead to a considerably increased stability of artificial colorings against the color being washed out. Chemically colored hair can thus be washed much more frequently using the agents as contemplated herein, without this leading to undesired bleeding or fading of the color.

Another subject matter as contemplated herein is therefore the use of agents as contemplated herein to reduce the washing-out of color from chemically colored hair.

With regard to preferred embodiments of the method as contemplated herein and of the use as contemplated herein, what has been stated above in respect of the agents as contemplated herein applies mutatis mutandis.

### Examples

All figures specified in % by weight

Hair Treatments

| | 1 | 2 | 3 | 4 | 5 | 6 |
|---|---|---|---|---|---|---|
| Cetearyl alcohol | 7.0 | 7.0 | 7.0 | 7.0 | 7.0 | 7.0 |
| Quaternium-87 | 0.9 | 0.9 | 0.9 | 0.9 | 0.9 | 0.9 |
| Cetrimonium chloride | 3.5 | 3.5 | 3.5 | 3.5 | 3.5 | 3.5 |
| Distearoylethyl | 2.0 | 2.0 | 2.0 | 2.0 | 2.0 | 2.0 |
| hydroxyethylmonium | | | | | | |
| methosulfate | | | | | | |
| Polyquaternium-10 | 0.1 | 0.1 | 0.1 | 0.1 | 0.1 | 0.1 |
| Dimethicone | 1.0 | 1.0 | 1.0 | 1.0 | 1.0 | 1.0 |
| Baobab seed oil | 0.1 | 0.1 | 0.1 | 0.1 | 0.1 | 0.1 |
| Polyquaternium-37 | 0.4 | 0.4 | 0.4 | 0.4 | 0.4 | 0.4 |
| Hydrolyzed keratin | 0.5 | 0.5 | 0.5 | 0.5 | 0.5 | 0.5 |
| Glycol distearate | 1.0 | 1.0 | 1.0 | 1.0 | 1.0 | 1.0 |
| Citric acid | 1.5 | 1.5 | 1.5 | 1.5 | 1.5 | 1.5 |
| Hexetidine | 0.06 | 0.06 | 0.06 | 0.06 | 0.06 | 0.06 |
| Dimethyloxazolidine | — | 0.05 | 0.06 | 0.07 | 0.08 | 0.1 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Phenoxyethanol | 0.3 | 0.3 | 0.3 | 0.3 | 0.3 | 0.3 |
| Phenoxyisopropanol | — | 1.0 | 0.5 | 0.25 | 0.2 | 0.1 |
| o-Cymen-5-ol | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Colorant | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | ad 100 |

Rinse-Out Hair Conditioners

| | 1 | 2 | 3 | 4 | 5 | 6 |
|---|---|---|---|---|---|---|
| Cetearyl alcohol | 5.0 | 5.0 | 5.0 | 5.0 | 5.0 | 5.0 |
| Quaternium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Behentrimonium | 1.0 | 1.0 | 1.0 | 1.0 | 1.0 | 1.0 |
| chloride | | | | | | |
| Behenoyl PG | 1.5 | 1.5 | 1.5 | 1.5 | 1.5 | 1.5 |
| trimonium chloride | | | | | | |
| Distearoylethyl | 0.3 | 0.3 | 0.3 | 0.3 | 0.3 | 0.3 |
| hydroxyethylmonium | | | | | | |
| methosulfate | | | | | | |
| Amodimethicone/ | 0.1 | 0.1 | 0.1 | 0.1 | 0.1 | 0.1 |
| morpholinomethyl | | | | | | |
| silsesquioxane | | | | | | |
| copolymer | | | | | | |
| Guar hydroxypropyl- | 0.4 | 0.4 | 0.4 | 0.4 | 0.4 | 0.4 |
| trimonium chloride | | | | | | |
| Lactic acid | 0.1 | 0.1 | 0.1 | 0.1 | 0.1 | 0.1 |
| Shea butter | 1.5 | 1.5 | 1.5 | 1.5 | 1.5 | 1.5 |
| Hexetidine | 0.1 | 0.1 | 0.1 | 0.1 | 0.1 | 0.1 |
| Dimethyloxazolidine | — | 0.1 | 0.1 | 0.1 | 0.1 | 0.1 |
| Ethyl lauroyl arginate | — | 0.05 | 0.06 | 0.07 | 0.08 | 0.1 |
| Glutaraldehyde | — | 0.4 | 0.3 | 0.2 | 0.1 | 0.05 |
| Phenoxyethanol | 0.3 | 0.3 | 0.3 | 0.3 | 0.3 | 0.3 |
| Phenoxyisopropanol | — | 1.0 | 0.5 | 0.25 | 0.2 | 0.1 |
| o-Cymen-5-ol | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Colorant | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | ad 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

1. A hair treatment agent, comprising:
a) at least one fatty alcohol,
b) at least one cationic surfactant,
c) hexetidine, and
d) phenoxyethanol.

2. The hair treatment agent according to claim 1 comprising a) fatty alcohol(s) of formula (II)
H3C—(CH2)k-CH2-OH (II)
in which k denotes integers from 4 to 28.

3. The hair treatment agent according to claim 1, comprising, based on the weight thereof, about 0.05 to about 20 wt. %, of b) cationic surfactant(s)

4. The hair treatment agent according to claim 1, comprising as b) behenyltrimethylammonium chloride as b).

5. The hair treatment agent according to claim 1, comprising, based on its weight, about 0.001 to about 0.3% by weight of c) hexetidine.

6. The hair treatment agent according to claim 1, comprising, based on its weight, about 0.01 to about 5% by weight of d) phenoxyethanol.

7. The hair treatment agent according to claim 1, further comprising at least one polymer chosen from the group of cationic cellulose polymers and/or cationic guar derivatives.

8. The hair treatment agent according to claim 1, further comprising at least one organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-napthalenepentaacetic acid, malonaldehydic acid, 4-hydroxyphthalamidic acid, 1-pyrazolecarboxylic acid, gallic acid or propanetricarboxylic acid, glycolic acid, lactic acid, malic acid, tartaric acid or citric acid.

9. The hair treatment agent according to claim 8, comprising at least one organic acid(s) chosen from the group of formic acid, oxalic acid, maleic acid, lactic acid, tartaric acid or citric acid.

10. A method for treating hair, comprising:
applying a hair treatment agent to dry or moist hair, wherein the hair treatment agent comprises:
a) at least one fatty alcohol,
b) at least one cationic surfactant,
c) hexetidine, and
d) phenoxyethanol,
where the hair treatment is agent is left on the hair for a period of about 30 to about 300 seconds and then rinsed off.

11. The hair treatment agent according to claim 2 comprising, based on the weight thereof, about 0.1 to about 20 wt. % of a) fatty alcohol(s) of formula (II).

12. The hair treatment agent according to claim 2, in which k denotes integers from 8 to 22.

13. The hair treatment agent according to claim 3, comprising, based on the weight thereof, about 0.5 to about 7 wt. % of b) cationic surfactant(s).

14. The hair treatment agent according to claim 4, comprising, based on its weight, about 0.05 to about 20% by weight of behenyltrimethylammonium chloride as b).

15. The hair treatment agent according to claim 7, comprising, based on the weight of the agent, about 0.01 to about 3% by weight of the at least one polymer chosen from the group of cationic cellulose polymers and/or cationic guar derivatives.

16. The hair treatment agent according to claim 8, comprising, based on its weight, about 0.001 to about 10% by weight of organic acid(s).
